(19) United States
(12) Patent Application Publication
(10) Pub. No.: US 2026/0357309 A1
(43) Pub. Date: Nov. 12, 2026

(54) METHOD FOR PRODUCING HYPOTAURINE BY FERMENTATION

(71) Applicant: Wacker Chemie AG, Munich (DE)

(72) Inventor: Rupert PFALLER, München (DE)

(73) Assignee: Wacker Chemie AG, Munich (DE)

(21) Appl. No.: 19/137,653

(22) PCT Filed: Dec. 15, 2022
(86) PCT No.: PCT/EP2022/086034

§ 371 Date: Jun. 11, 2025

(51) Int. Cl. C12P 13/00

(52) U.S. Cl. CPC C12P 13/001

(57) ABSTRACT

The invention relates to a process for fermentative production of hypotaurine. In a step i, a microbial production strain is cultivated. The microbial production strain is distinguished in that it contains at least one expression vector comprising a coding sequence (cds) a and a cds b. In a step ii, the fermentation supernatant is isolated, the hypotaurine content in the fermentation supernatant is at least 10 g/L, and the molar fraction of hypotaurine:taurine in the fermentation supernatant being at least 3:1.

The invention relates to a process for fermentative production of hypotaurine, characterized in that
i) a microbial production strain is cultivated, said microbial production strain being distinguished in that it
   1. contains at least one expression vector comprising a coding sequence (cds) a and a cds b and at least one of the cds selected from the group consisting of c, d and e,
      a) a being a cds encoding a cysteine dioxygenase (CDO) belonging to enzyme class EC 1.13.11.20 and
      b) b being a cds encoding a cysteine sulfinic acid decarboxylase (CSAD) belonging to enzyme class EC 4.1.1.29 and
      c) c being a cds encoding a 3-phosphoglycerate dehydrogenase (SerA) in which feedback inhibition by serine is reduced by a factor of at least two relative to the corresponding wild-type enzyme and
      d) d being a cds encoding a serine O-acetyltransferase (CysE) in which feedback inhibition by cysteine is reduced by a factor of at least two relative to the corresponding wild-type enzyme and
      e) e being a cds encoding a cysteine exporter,
   2. the expression of the cds a and b in the expression vector is coordinated with the expression of at least one cds selected from the group consisting of c, d and e in a polycistronic expression unit,
   3. hypotaurine is secreted by the production strain into the fermentation supernatant, and
ii) the fermentation supernatant is isolated,
the hypotaurine content in the fermentation supernatant being at least 10 g/L and the molar fraction of hypotaurine:taurine in the fermentation supernatant being at least 3:1.

Hypotaurine (2-aminoethanesulfinic acid, CAS number 300-84-5) is an aminosulfinic acid that occurs in nature as a breakdown product of the amino acid cysteine. As a sulfinic acid, hypotaurine acts as an antioxidant and is used in the food, feed, cosmetics and pharmaceutical sectors. Furthermore, hypotaurine is a biosynthetic precursor of taurine (2-aminoethanesulfonic acid, CAS number 107-35-7).

Hypotaurine for commercial use is produced chemically, for example from 2-aminoethanethiol hydrochloride as the starting point. Since there is a consumer-driven trend away from chemically produced ingredients, a biotechnological process for the production of hypotaurine is of interest. Known biosynthetic pathways to 5 hypotaurine and onward to taurine, starting from L-cysteine, can be found for example in the KEGG Pathway Database: “Taurine and hypotaurine metabolism”.

The most important synthetic steps leading from L-cysteine to hypotaurine and onward to taurine are shown in equations (1) to (5).

L-Cysteine+O₂->L-Cysteine sulfinic acid (1)

L-Cysteine->Cysteamine+CO₂ (2)

L-Cysteine sulfinic acid->Hypotaurine+CO₂ (3)

Cysteamine+O₂->Hypotaurine (4)

Hypotaurine+½O₂->Taurine (5)
(1): L-cysteine is oxidized by the enzyme cysteine dioxygenase (CDO, EC 1.13.11.20) to L-cysteine sulfinic acid (3-sulfinoalanine, CAS number 207121-48-0).
(2): Cysteamine is formed formally by decarboxylation of L-cysteine, but in mammals it is a reaction product of pantothenate biosynthesis, with the enzyme pantetheine hydrolase (EC 3.5.1.92) cleaving off cysteamine from the precursor (R)-pantetheine. A bacterial cysteine decarboxylase has been described, but it only acts on the C-terminal cysteine of a precursor peptide as a subreaction in the biosynthesis of a natural substance and is not suitable for the production of cysteamine. The pathway (2) to hypotaurine, starting from cysteine and via cysteamine, is thus of no technical significance.
(3): Cysteine sulfinate decarboxylase (CSAD, EC 4.1.1.29) decarboxylates L-cysteine sulfinic acid to hypotaurine (2-aminoethanesulfinic acid, CAS number 300-84-5).
(4): Cysteamine dioxygenase (EC 1.13.11.19) oxidizes cysteamine with atmospheric oxygen to hypotaurine.
(5): The oxidation of hypotaurine to taurine is not yet entirely clear.

Known biotechnological processes are primarily aimed at the production of taurine, but the route of the biosynthetic pathway means that hypotaurine is the main product in many cases. The known biotechnological processes for producing hypotaurine are characterized in that the hypotaurine yields, where disclosed, are very low.

Honjoh et al. (2010), Amino Acids 38: 173-1183 describe a genetically engineered yeast strain heterologously expressing the CDO gene and CSAD gene from carp (Cyprinus carpio). When L-cysteine was added to growth of the genetically engineered strain, what was observed was the production of hypotaurine as the main product and additionally a relatively low proportion of taurine. Even though S. cerevisiae itself is capable of producing cysteine from its own metabolism, the production of hypotaurine required the external addition of L-cysteine to the growth medium. The approach by Honjoh et al. (2010) thus equates to a biotransformation of L-cysteine to hypotaurine. Analytical determination of intracellularly formed taurine, with and without H₂O₂ treatment, was carried out (Table 1 in Honjoh et al.). From the distinctly higher taurine values following H₂O₂ treatment, it was inferred that a large portion of the intracellularly formed product was in the form of hypotaurine, even though analytical quantification of hypotaurine was not carried out. The indirect quantification of hypotaurine is surprising, since hypotaurine analysis was listed in the methods section of Honjoh et al. Thus, the formation of hypotaurine in baker's yeast has not been unambiguously demonstrated and was only indirectly inferred from the elevated taurine values following H₂O₂ oxidation.

WO 17/213142 A1 (Ajinomoto) describes taurine-producing strains as a result of heterologous expression of genes encoding cysteine dioxygenase (CDO) and L-cysteine sulfinic acid decarboxylase (CSAD) in a strain originally producing cysteine. Constructs composed of different CDO and CSAD genes were produced, each under the control of the known strong tac promoter. However, the suitability thereof for hypotaurine or taurine production was not carried out in strains optimized for cysteine production, but in strains of E. coli (maximum production of 2 μM cysteine; see Table 5 of WO 17/213142 A1, strain EcoT/pMW2q19) and Pantoea anananatis (maximum production of 0 μM cysteine; see Table 5 of WO 17/213142 A1, strain PanT/pMW219), each with an inactivated tauABCD operon (known as EcoT for E. coli and PanT for P. ananatis). The tauABCD operon is known to encode genes for the breakdown of taurine. The main product was hypotaurine with a maximum yield of 450 μM (49.1 mg/L hypotaurine with the molecular weight of hypotaurine being 109.2 g/mol). Thus, the optimization of cysteine metabolism as an option for improved hypotaurine production was described, but not experimentally demonstrated. The low hypotaurine yields do not allow economically viable production.

US 2019/0062757 A1 (KnipBio) describes heterologous production strains for the production of taurine or its precursors, the maximum yields achieved being only 419 ng/ml hypotaurine. The host strains used were WT strains of E. coli and Methylobacterium extorquens. Measures to improve cysteine production with the goal of higher hypotaurine yields were not described.

U.S. Pat. No. 9,267,148 B2 and other publications by Plant Sensory Systems describe gene constructs intended to be suitable for the heterologous expression of a cysteine dioxygenase and a L-cysteine sulfinic acid decarboxylase, the goal of which is the production of taurine. In particular, the applications relate, firstly, to gene constructs expressing (monocistronically in each case) a CDO gene and a CSAD gene, each functionally linked to its own promoter, as single expression cassettes. Secondly, also described are gene constructs which are intended to express a CDO gene and a CSAD gene in the form of a fusion protein, combined as a single expression cassette under the control of only one promoter (monocistronic expression cassette). The main focus of the applications is the production of taurine in plants, though nothing is said about yields. Measures to improve cysteine production with the goal of higher hypotaurine yields were not described. Altogether, the applications do not disclose any applicable method for fermentative production of hypotaurine.

Joo et al. (2018), J. Agric. Food Chem. 66: 13454-13463, describe a genetically engineered strain of the bacterium Corynebacterium glutamicum with an output of 0.5 g/L taurine when grown in a shake flask. It was apparent that taurine accumulated intracellularly and was not secreted into the growth medium. The synthesis of taurine was achieved by heterologously expressing the genes of an L-cysteic acid synthase, a cysteine dioxygenase and an L-cysteine sulfinic acid decarboxylase in the strain. In addition, a repressor gene in relation to methionine and cysteine biosynthesis was inactivated, which simultaneously achieved improved sulfur uptake. Analysis of the production of hypotaurine was not carried out.

The prior art thus discloses various metabolic engineering and biotransformation approaches for producing hypotaurine in heterologous production systems. Production strains having optimized cysteine biosynthesis were not used. None of the strains described in the prior art were disclosed in relation to the production of hypotaurine or taurine on an industrial scale by a fermentation process.

It is an object of the present invention to provide a process for fermentative production of hypotaurine with high yields in the fermentation supernatant. Since, according to the prior art, hypotaurine can oxidize to taurine, it is a further object to achieve a yield of hypotaurine that substantially exceeds that of taurine.

The object is achieved by a process for fermentative production of hypotaurine, characterized in that
i) a microbial production strain is cultivated, said microbial production strain being distinguished in that it
   1. contains at least one expression vector comprising a coding sequence (cds) a and a cds b and at least one of the cds selected from the group consisting of c, d and e,
      a) a being a cds encoding a cysteine dioxygenase (CDO) belonging to enzyme class EC 1.13.11.20 and
      b) b being a cds encoding a cysteine sulfinic acid decarboxylase (CSAD) belonging to enzyme class EC 4.1.1.29 and
      c) c being a cds encoding a 3-phosphoglycerate dehydrogenase (SerA) in which feedback inhibition by serine is reduced by a factor of at least two relative to the corresponding wild-type enzyme and
      d) d being a cds encoding a serine O-acetyltransferase (CysE) in which feedback inhibition by cysteine is reduced by a factor of at least two relative to the corresponding wild-type enzyme and
      e) e being a cds encoding a cysteine exporter,
   2. the expression of the cds a and b in the expression vector is coordinated with the expression of at least one cds selected from the group consisting of c, d and e in a polycistronic expression unit,
   3. hypotaurine is secreted by the production strain into the fermentation supernatant, and
ii) the fermentation supernatant is isolated,
the hypotaurine content in the fermentation supernatant being at least 10 g/L and the molar fraction of hypotaurine:taurine in the fermentation supernatant being at least 3:1.

A major advantage of the present invention is that a fermentative process for producing hypotaurine is disclosed, in which the expression vectors designed according to the principle of metabolic engineering combine the expression units for increased cysteine production with expression units for hypotaurine production in the microbial production strain. Especially the polycistronic arrangement of at least one cds of cysteine metabolism with the cds for cysteine dioxygenase (CDO) and the cds for cysteine sulfinic acid decarboxylase (CSAD) in an artificial operon under the control of just one promoter allows the coordinated expression of cysteine and hypotaurine metabolism genes in a production strain. This helps to avoid the accumulation of metabolic intermediates, for example L-cysteine, and maximizes the yield of hypotaurine.

It is surprising that the product hypotaurine is secreted into the fermentation supernatant (extracellular production) by the production strain in a concentration of ≥10 g/l and accumulates extracellularly (Example 5). In the prior art by contrast, what occurs is intracellular enrichment, as described for taurine by Joo et al. (2018, see above) for example, or, depending on the production system, only partial secretion.

The use of the production strain according to the claims in the process according to the invention is thus distinguished by the lack of accumulation of product in the biomass (fermenter cells) (see also Example 5). This extracellular production or enrichment, i.e., the biosynthesis of hypotaurine in the production strain followed by its secretion (export, transfer) out of the production strain into the growth medium, has the major advantage that the volume in which hypotaurine can accumulate is not limited to the small volume of cell content (cystosol). This avoids toxic effects due to high concentrations of intracellular product. Therefore, far higher hypotaurine yields can be achieved than with intracellular production. Preferably, the biomass contains less than 1 g/l hypotaurine, based on the biomass present in the fermenter broth, as described in Example 5. Particularly preferably, there is no detectable hypotaurine in the biomass.

In addition, the extracellular production of hypotaurine has the advantage of simplified product isolation, since hypotaurine can be isolated directly from the fermentation supernatant without any need for elaborate mechanical or chemical disruption of the cells beforehand. It is not known how hypotaurine is secreted out of the production strain. The mechanism may be passive, with diffusion of hypotaurine through the cell membranes, or one or more transport proteins may be involved in secretion.

Another advantage of the present invention is the low proportion of the by-product taurine, which is formed from hypotaurine by oxidation and reduces the yield of hypotaurine. Thus, the process according to the invention is very highly suited to industrial use, since it allows the economically viable production of hypotaurine thanks to the high yields of hypotaurine in fermentation. By contrast, in the prior art such as Joo et al. (2018, see above), spontaneous oxidation of hypotaurine to taurine occurs, which has the disadvantage of a low hypotaurine content.

Metabolic engineering (also called “pathway design”), in contrast to biotransformation, is a biotechnology method in which metabolic pathways of an organism are modified by optimization or modification of genetic and regulatory processes. New or modified enzymes can be introduced into an organism by supplementation of the genome with genes of enzymes, or genes of endogenous enzymes can be expressed at an enhanced or attenuated level, thereby establishing new metabolic pathways in an organism or enhancing or attenuating existing metabolic pathways. The goal of metabolic engineering is for the organism to produce either a new metabolite or a cell-endogenous metabolite at increased yield. A metabolic engineering process does not use starting materials specific for the metabolite, such as an enzyme substrate, for example L-cysteine as starting compound for the production of hypotaurine; instead, it only uses a nutrient medium, also referred to as growth medium, which is required for the growth of the organism in question and is composed of a carbon source (e.g., glucose), a nitrogen source (e.g., an ammonium salt or a complex amino acid mixture such as peptone or yeast extract) and other salts required for growth. Such nutrient media are known to a person skilled in the art from microbiological practice. The process disclosed in the present invention for fermentative production of hypotaurine is a metabolic engineering process.

By contrast, biotransformation is defined as the transformation of one or more reactants into a product under enzymatic catalysis, the enzyme substrate being added with the enzyme to a reaction and enzymatically converted.

According to equation (1), L-cysteine is enzymatically oxidized to L-cysteine sulfinic acid. This reaction is catalyzed by a CDO enzyme (EC 1.13.11.20). Gene and protein sequences of CDO enzymes are, for example, available in the NCBI database under the search term “cysteine dioxygenase”. The invention encompasses genes encoding proteins having CDO activity, including preferably CDO enzymes selected from Rattus norvegicus (rat), Homo sapiens (human), Cyprinus carpio (carp), Bos taurus (cattle), Capra hircus (goat), Gallus gallus (chicken) or Synechococcus (alga) or homologous CDO enzymes. In a preferred embodiment, the process is characterized in that the CDO is the CDO from Rattus norvegicus (CDOrn) having the sequence specified in SEQ ID NO: 2 or an at least 80%, particularly preferably at least 90% and especially preferably at least 95% identical enzyme having CDO activity. Especially preferably, the CDO is a protein having SEQ ID NO: 2. Specially preferably, the CDOrn cds has the sequence specified in SEQ ID NO: 1.

The CDO enzyme activity assay may be carried out as follows:
i) producing the enzyme to be tested:
   An enzyme produced by growth in a shake flask or in a fermentation may be used in the reaction as follows:
      as an aliquot from the culture broth without further workup or
      as an aliquot of the cell suspension after reisolation of the cells from the culture broth, for example by centrifugation, or
      in the form of an aliquot of the cell homogenate
      a) after mechanical disruption of the cell suspension or
      b) in the form of chemically permeabilized cells (e.g., by chloroform) or
      as a cell extract after removal of particulate constituents from the cell homogenate or
      as an enzyme purified by chromatography, for example.
   The total protein concentration obtained in each case can be determined, for example, by means of a commercially available Qubit 3.0 Fluorometer from Thermo Fisher Scientific using the “Qubit® Protein Assay Kit” according to the manufacturer's instructions.
ii) determining CDO enzyme activity:
   A solution buffered to pH 7 with potassium phosphate is initially charged with L-cysteine (10 mM final concentration) and the reaction is started by addition of the enzyme from i). The assay volume is 10 ml. The temperature at which the assay is carried out is 30 °C. The amount of enzyme used depends on the purity. If culture broth, cell suspension of reisolated cells, cell homogenate or cell extract is used, at least 0.1 mg of the enzyme fractions prepared in i) is used. In the case of purified enzyme, at least 10 μg of the purified enzyme fraction are used. 1 h, 2 h and 4 h after the start of the reaction, 1 ml of the assay is removed in each case and centrifuged for 10 min and the content of L-cysteine sulfinic acid is determined by calibrated HPLC (see Example 3); the reference substances used for calibration are commercially available (Sigma-Aldrich).

The detection limit of the HPLC method is 1 mg/L L-cysteine sulfinic acid. If less than 1 mg/L L-cysteine sulfinic acid is formed under the aforementioned assay conditions, then the assay does not contain any active CDO.

According to equation (3), L-cysteine sulfinic acid is enzymatically decarboxylated to hypotaurine. This reaction is catalyzed by a CSAD enzyme (EC 4.1.1.29) or alternatively, with far lower enzyme activity, GAD enzyme (EC 4.1.1.15). Gene and protein sequences of CSAD enzymes are, for example, available in the NCBI database under the search term “cysteine sulfinic acid decarboxylase”. Gene and protein sequences of GAD enzymes are, for example, available in the NCBI database under the search term “glutamic acid decarboxylase”. The invention encompasses genes encoding proteins having CSAD activity, including preferably CSAD enzymes selected from Cyprinus carpio (carp), Rattus norvegicus (rat), Homo sapiens (human), Bos taurus (cattle), Capra hircus (goat), Gallus gallus (chicken), Escherichia coli or Synechococcus (alga) or homologous CSAD enzymes. In a preferred embodiment, the process is characterized in that the CSAD is the CSAD from Cyprinus carpio (CSADcc) having the sequence specified in SEQ ID NO: 6 or an at least 80%, particularly preferably at least 90% and especially preferably at least 95% identical enzyme having CSAD activity. Especially preferably, the CSAD is a protein having SEQ ID NO: 6. Specially preferably, the CSADcc cds has the sequence specified in SEQ ID NO: 5 (nt 1-1503).

In an alternatively preferred embodiment, the process is characterized in that the CSAD is the CSAD from Homo sapiens (CSADhs) having the sequence specified in SEQ ID NO: 4 or an at least 80%, particularly preferably at least 90% and especially preferably at least 95% identical enzyme having CSAD activity.

Especially preferably, the CSAD is a protein having SEQ ID NO: 4. Specially preferably, the CSADhs cds has the sequence specified in SEQ ID NO: 3 (nt 1-1509).

The CSAD enzyme activity assay may be carried out as follows:
i) producing the enzyme to be tested:
   An enzyme produced by growth in a shake flask or in a fermentation may be used in the reaction as follows:
      as an aliquot from the culture broth without further workup or
      as an aliquot of the cell suspension after reisolation of the cells from the culture broth, for example by centrifugation, or
      in the form of an aliquot of the cell homogenate
      a) after mechanical disruption of the cell suspension or
      b) in the form of chemically permeabilized cells (e.g., by chloroform) or
      as a cell extract after removal of particulate constituents from the cell homogenate or
      as an enzyme purified by chromatography, for example.
   The total protein concentration obtained in each case can be determined, for example, by means of a commercially available Qubit 3.0 Fluorometer from Thermo Fisher Scientific using the “Qubit® Protein Assay Kit” according to the manufacturer's instructions.
ii) determining CSAD enzyme activity:
   A solution buffered to pH 7 with potassium phosphate is initially charged with L-cysteine sulfinic acid (10 mM final concentration) and the reaction is started by addition of the enzyme from i). The assay volume is 10 ml. The temperature at which the assay is carried out is 30 °C. The amount of enzyme used depends on the purity. If culture broth, cell suspension of reisolated cells, cell homogenate or cell extract is used, at least 0.1 mg of the enzyme fractions prepared in i) is used. In the case of purified enzyme, at least 10 μg of the purified enzyme fraction are used. 1 h, 2 h and 4 h after the start of the reaction, 1 ml of the assay is removed in each case and centrifuged for 10 min and the content of hypotaurine is determined by calibrated HPLC (see Example 3); the reference substances used for calibration are commercially available (Sigma-Aldrich).

The detection limit of the HPLC method is 1 mg/L hypotaurine. If less than 1 mg/L hypotaurine is formed under the aforementioned assay conditions, then the assay does not contain any active CSAD.

Open reading frame (ORF, synonymous with cds or coding sequence) refers to a region of DNA or RNA that begins with a start codon and ends with a stop codon and encodes the amino acid sequence of a protein. The ORF is also referred to as the coding region or structural gene.

Gene, cistron or expression unit refers to the section of DNA that contains all the basic information for producing a biologically active RNA. A gene contains the section of DNA from which a single-stranded RNA copy is produced by transcription and also the expression signals involved in the regulation of this copying process.

The expression signals include at least a promoter, a transcription start, a translation start, and a ribosome binding site (RBS). A terminator and one or more operators are additional possible expression signals.

In the context of this invention, bacterial proteins, for example SerA or CysE, start with a capital letter, whereas the sequences encoding said proteins (cds) are identified by a lowercase letter (e.g., serA or cysE). Similarly, the promoters controlling the expression of said cds are identified by a lowercase letter (e.g., serA promoter or cysE promoter). By contrast, proteins/enzymes and cds/genes of higher organisms, for example Homo sapiens, Rattus norvegicus or Cyprinus carpio, are identified by capital letters and, if necessary, are labelled as a protein/enzyme (e.g., CDO protein/enzyme or CSAD protein/enzyme) or cds/gene (e.g., CDO cds/gene or CSAD cds/gene) in each case.

Gene construct refers to a DNA molecule produced by cloning that comprises at least one expression unit and may also comprise other genetic elements alongside it, such as a selection marker and origin of replication. The gene construct may be a linear DNA molecule integrated into the genome or it may be a circular DNA molecule in the form of a plasmid, also called synonymously a vector. Said vector is then referred to as an expression vector. After insertion (transformation) into a suitable host strain, the genetic elements of the vector bring about the extrachromosomal inheritance of said vector during cell growth and bring about the production of the protein encoded by the cds.

In the genomes of microorganisms, so-called operons can be found in many cases. An operon is characterized by a section of DNA from which multiple genes can be expressed in a coordinated manner. Operon refers to a section of DNA that contains all the basic information for producing a biologically active RNA. The operon contains the section of DNA from which a single-stranded RNA copy is produced by transcription and also the expression signals involved in the regulation of this copying process. However, the RNA copy comprises not just the cds of a single gene, but the cds of two or more genes. The expression signals of the operon include a promoter and a transcription start. Each cds of the operon includes a translation start and a ribosome binding site. A terminator and one or more operators are additional possible expression signals.

If only one gene is expressed by one promoter, this is referred to as a monocistronic expression unit. If two, three or more genes are expressed by one promoter, then such an expression unit is referred to as bicistronic, tricistronic, and so forth, or polycistronic.

Operons not only occur naturally in the genome of microorganisms, but can also be produced specifically by cloning (artificial operons).

The expression unit of an artificial operon likewise consists of one promoter functionally linked to two or more cds with an upstream RBS in each case (bi-, tri- or polycistronic expression unit). In addition, an expression unit may also contain other genetic elements, such as a terminator or operator. The term “functionally linked” means that the expression unit comprising promoter and cds, or promoter and two or more cds in the case of an operon, leads to the transcription and translation of the cds in the microorganism.

Promoter refers to a nucleotide sequence that allows the expression of a cds and is upstream of the 5′ end of the cds. In the direction of synthesis, the promoter comes before the coding region. The promoter contains regions that can define the start of transcription of the gene by the RNA polymerase and additionally mediate the specific interaction with DNA-binding proteins influencing the level of transcription (transcription factors).

All promoters active in the host strain are generally suitable as promoters of the aforementioned cds. These include all native promoters of the approximately 5000 genes in E. coli, but also non-native promoters (e.g., promoters from other species of the Enterobacteriaceae family) or “artificial” promoters such as the tac promoter.

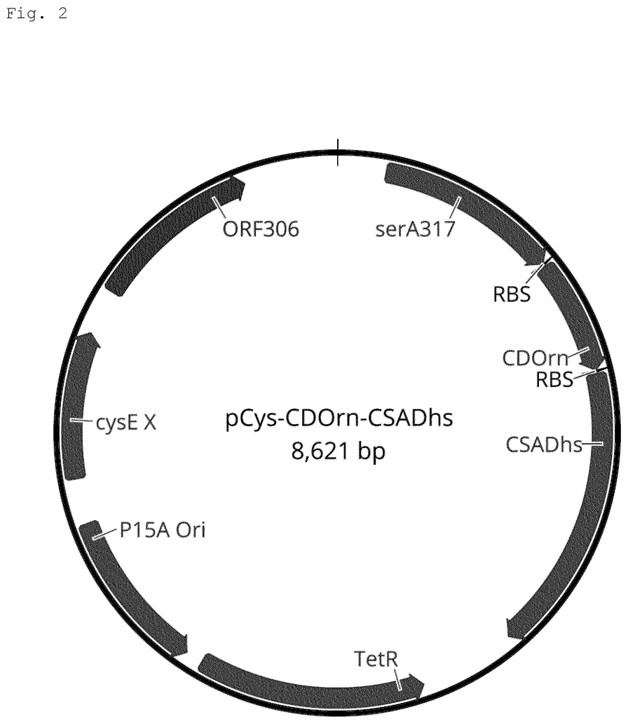

Preferred promoters in the polycistronic expression unit are the cysE, serA and GAPDH promotors present on pCys, particularly preferably the serA promoter, as described for example in Rex et al. (1991), J. Bacteriol. 173: 5944-5953, FIG. 2, nt 1-nt 457.

Especially preferred is the serA promoter having the sequence specified in SEQ ID NO: 7.

An mRNA, also known as messenger RNA, is a single-stranded ribonucleic acid (RNA) that carries the genetic information for the synthesis of a protein. An mRNA provides the assembly instructions for a particular protein in a cell. The mRNA molecule conveys the requisite message for protein synthesis from the genetic information (DNA) to the ribosomes responsible for protein synthesis. In a cell it is formed as a transcript of a section of DNA corresponding to a gene. The genetic information stored in the DNA is unchanged by this process.

Genes of eukaryotic organisms are predominantly what are known as mosaic genes and, unlike prokaryotic genes, also contain non-coding sections known as introns (intragenic regions). Coding sequences, which are known as exons (expressed regions), are sections of DNA of a eukaryotic gene that, after being transcribed into RNA, are translated by the ribosomes into the amino acid sequence of a protein.

After transcription of DNA into RNA, the introns are spliced from the primary transcript. The protein-coding RNA with introns removed is referred to as messenger RNA (mRNA) or “mature” mRNA. This undergoes further modifications such as capping and polyadenylation. The coding region of the mature mRNA is then translated into the protein sequence. If a eukaryotic gene containing an exon/intron structure is to be expressed in prokaryotic organisms, it is necessary to back-translate the protein sequence or coding region of the mature mRNA into intron-free DNA, since no processing of the exon/intron structure takes place in prokaryotes. Whenever the expression gene sequences derived from the protein sequence or gene sequences derived from mRNA is used in the context of this invention, it is precisely this process of back-translation that is meant. It is preferable that sequence optimization, i.e. adaptation to the codon usage of the corresponding prokaryote (codon optimization), takes place concomitantly with the back-translation of the protein sequence or the mRNA sequence into a DNA sequence.

Homologous genes or homologous DNA sequences are to be understood to mean that the DNA sequences of these genes or sections of DNA are at least 80% identical, preferably at least 90% identical and particularly preferably at least 95% identical.

The degree of DNA identity is determined by the “nucleotide blast” program, which can be found at http://blast.ncbi.nlm.nih.gov/ and is based on the blastn algorithm.

The algorithm parameters used to align two or more nucleotide sequences were the default parameters. The default general parameters are: Max target sequences=100; Short queries=“Automatically adjust parameters for short input sequences”; Expect Threshold=10; Word size=28; Automatically adjust parameters for short input sequences=0. The corresponding default scoring parameters are: Match/Mismatch Scores=1,−2; Gap Costs=Linear.

Homologous protein sequences are to be understood to mean that the protein sequences of these proteins or sections of protein are at least 80% identical, preferably at least 90% identical and particularly preferably at least 95% identical.

Protein sequences are compared using the “protein blast” program at http://blast.ncbi.nlm.nih.gov/. This program uses the blastp algorithm. The algorithm parameters used to align two or more protein sequences were the default parameters. The default general parameters are: Max target sequences=100; Short queries=“Automatically adjust parameters for short input sequences”; Expect Threshold=10; Word size=3; Automatically adjust parameters for short input sequences=0. The default scoring parameters are: Matrix=BLOSUM62; Gap Costs=Existence: 11 Extension: 1; Compositional adjustments=Conditional compositional score template adjustment.

The abbreviation WT (Wt) refers to the wild type. Wild-type gene refers to the form of the gene that arose naturally through evolution and is present in the wild-type genome. The DNA sequence of Wt genes is publicly available in databases such as NCBI.

Alleles define the states of a gene that can be converted into one another by mutation, i.e., by changes to the nucleotide sequence of the DNA. The gene that occurs naturally in a microorganism is referred to as the wild-type allele and the variants derived therefrom are referred to as the mutated alleles of the gene.

Fermentation is a process step for the production (cultivation) of cell cultures on an industrial scale, in which a preferably microbial production strain is made to grow under defined conditions of culture medium, temperature, pH, oxygen supply, and mixing of the medium. If all the fermentation components are defined at the beginning of cultivation and then no longer changed, the fermentation is called a batch fermentation.

If, after the start of fermentation, media constituents such as glucose (carbon source) or a complex amino acid mixture such as yeast extract (nitrogen source) are supplied continuously as a so-called feed, the fermentation is called a fed-batch fermentation (so-called feed process). Fed-batch fermentation allows the optimization of the formation of biomass and target product. Depending on the configuration (genetic makeup) of the production strain, the aim of fermentation is to produce the highest possible yield of a protein/enzyme or a metabolite for further use. The product hypotaurine may be produced by fermentation. The final product of fermentation is a fermenter broth consisting of the biomass of the cells of the production strain (fermenter cells) and of the biomass-free fermentation medium (fermentation supernatant) that formed over the course of fermentation from the growth medium and from the metabolites secreted by the fermenter cells. In contrast to the growth medium defined by its chemical composition, the composition of the fermentation medium is not clearly defined, since the formation of metabolites is not predictable. In the present invention, the product hypotaurine is produced by fermentation. In principle, the products of fermentation may be present in the fermenter cells and/or in the fermentation medium. As disclosed in Example 5 of the present invention, hypotaurine is found exclusively in the fermentation medium within the detection limit. Therefore, only the hypotaurine produced by secretion into the fermentation medium is taken into account.

Preferably, the process is characterized in that the hypotaurine content in the fermentation supernatant based on the hypotaurine content in the fermentation batch is above 70%, preferably above 80% and especially preferably above 90%. The hypotaurine content in the fermentation supernatant corresponds to the extracellular (secreted) hypotaurine. The hypotaurine content in the fermentation batch is the sum total of extracellular and intracellular hypotaurine content, also referred to as total hypotaurine content. The extracellular portion of hypotaurine is determined as described in Example 3 (“Sample preparation”). The intracellular portion of hypotaurine is determined as described in Example 5, the detection limit for hypotaurine being 1 mg/L.

Shake flask growth is used to cultivate microorganisms on a laboratory scale, in contrast to a fermentation production scale. Although a shake flask culture also involves specifying a particular medium and a pH and cultivating in the presence of oxygen under constant motion (shaking), more defined conditions concerning the medium, temperature, pH, oxygen supply, and mixing of the medium can be established and regulated in a fermenter. A smaller-scale culture, for example in a shake flask, may also be used as a preculture for inoculation of a larger-scale culture, for example a fermenter.

The production scale in fermentation typically starts at a batch volume of 0.5 L and may be up to 100 000 L or higher. The production scale of shake flask growth typically ranges from 10 ml to 1000 ml in batch volume.

Yield in the context of the invention is defined as the amount of product obtained by growing a production strain. The yield can be specified in an absolute amount of product (mmol or g) or as a volume yield (concentration) in terms of amount of product based on the volume (mM or g/L).

The process according to the invention comprises a fermentative process for producing hypotaurine using a microbial production strain. By definition, a production strain comprises a microorganism, referred to a host strain, and at least one gene construct. In the present invention, the production strain is characterized in that the gene construct is an expression vector.

A suitable host strain is any microorganism that is amenable to recombinant DNA techniques and that is suitable for fermentative production of recombinant proteins.

Suitable microorganism strains include bacterial strains selected from the Corynebacteriaceae family or the Enterobacteriaceae family, yeasts (e.g., Saccharomyces cerevisiae, Yarrowia lipolytica) or fungi (e.g., Aspergillus niger).

The host strain used for producing the production strain is preferably a prokaryotic microorganism, particularly preferably a bacterial strain selected from the group consisting of Corynebacterium ssp. (such as particularly preferably C. glutamicum), Pantoea ssp. (such as particularly preferably P. ananatis) and Escherichia ssp., and especially preferably a microorganism of the species Escherichia coli. In a specially preferred embodiment, the microorganism is the strain E. coli K12 W3110, commercially available under the strain number DSM 5911 from the DSMZ-German Collection of Microorganisms and Cell Cultures GmbH.

A production strain for fermentative production of at least 10 g/l hypotaurine is characterized in that it contains a gene construct encompassing
(I) a metabolic pathway which leads to hypotaurine and is defined by the expression of the cds a (encoding CDO) and b (encoding CSAD) and
(II) a deregulated cysteine biosynthesis pathway defined by the expression of at least one of the cds selected from the group consisting of c (encoding SerA having reduced feedback inhibition by serine), d (encoding CysE having reduced feedback inhibition by cysteine) and e (encoding a cysteine exporter).

(I) The metabolic pathway which leads to hypotaurine is characterized in that it comprises the conversion of L-cysteine to hypotaurine according to equations (1) and (3).

The genes CDO and CSAD in the production strain that constitute the hypotaurine metabolic pathway may stem from the microorganism strain that is also the host strain (homologous expression) or they may be foreign genes (heterologous expression) that are either produced by synthesis (which allows, inter alia, adaptation of the cds by so-called codon optimization for expression in the host strain) or isolated from strains other than the host strain, or a combination of homologous and heterologous expression may be carried out.

Preference is given to the heterologous expression of the genes of the hypotaurine-constituting metabolic pathway in the production strain, particular preference is given to the heterologous expression of synthetically produced genes, and especial preference is given to the heterologous expression of codon-optimized, synthetically produced genes of the hypotaurine-constituting metabolic pathway in the production strain.

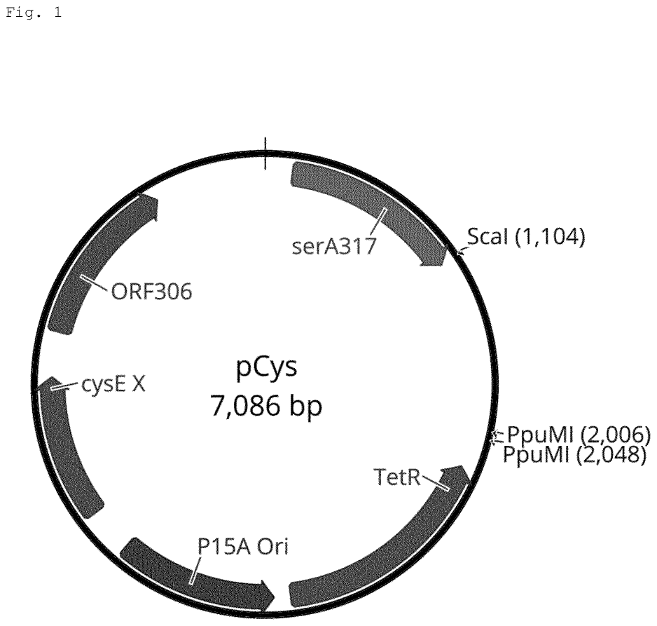

(II) A production strain containing a gene construct comprising expression units for a deregulated cysteine biosynthesis pathway and thus suitable for cysteine production is characterized in that it contains at least one of the cds selected from the group consisting of c, d and e:
c) The production strain comprises at least one modified serA gene, i.e., it contains at least one cds c encoding a 3-phosphoglycerate dehydrogenase (SerA) in which feedback inhibition by L-serine is reduced by a factor of at least two relative to the corresponding wild-type enzyme, where the SerA enzyme activity can be determined for example photometrically through the oxidation of NADH dependent on the SerA substrate 3-phosphohydroxypyruvate, as described for example by McKitrick and Pizer, J. Bacteriol. (1980) 141: 235-245. Furthermore, the production strain may additionally contain the unmodified WT serA gene.
   In particularly preferred variants of the 3-phosphoglycerate dehydrogenase (SerA), feedback inhibition by L-serine is reduced by a factor of at least 5 relative to the corresponding wild-type enzyme, especially preferably by a factor of at least 10 and, in a more preferred embodiment, by a factor of at least 50. Examples of serA variants encoding SerA enzymes having reduced feedback inhibition are known from the prior art and are preferred embodiments in the context of this invention:
      serA317: Feedback-resistant SerA mutant as used in the vector pCys (FIG. 1) in the present invention. It is a C-terminal deletion mutant of SerA comprising the N-terminal 317 amino acids of the SerA WT protein with a total length of 410 amino acids (Bell et al., Eur. J. Biochem., 2002, 269: 4176-4184, referred to therein as “NSD:317”; see also Example 1).
      serA G349E: Mutation of glycine at position 349 of the WT SerA enzyme to glutamic acid.
      serA G349D: Mutation of glycine at position 349 of the WT SerA enzyme to aspartic acid.
      serA T372X: Mutation of threonine at position 372 of the WT SerA enzyme to X, where X can be any natural amino acid except threonine.
      serA mutated: serA gene in which 25% of the C-terminal amino acids are modified compared to the WT, the modification being
      i) in the 50 C-terminal amino acids of the WT protein and
      ii) the modification comprising a C-terminal deletion and
      iii) the modification comprising an insertion into the WT sequence. and/or
d) The production strain comprises at least one modified cysE gene, i.e., it contains at least one cds d encoding a serine O-acetyltransferase (CysE) in which feedback inhibition by cysteine is reduced by a factor of at least two relative to the corresponding wild-type enzyme (as described for example in Nakamori et al., Appl. Env. Microbiol. (1998) 64: 1607-1611), where the CysE enzyme activity can be determined for example photometrically through the consumption of the CysE substrate acetyl-CoA resulting from the reaction with L-serine to form O-acetyl-L-serine, as described for example by Nakamori et al., Appl. Env. Microbiol. (1998) 64: 1607-1611). Furthermore, the production strain may additionally contain the unmodified WT cysE gene.
   In particularly preferred variants of the serine O-acetyltransferase (CysE), feedback inhibition by cysteine is reduced by a factor of at least 5 relative to the corresponding wild-type enzyme, especially preferably by a factor of at least 10 and, in a more preferred embodiment, by a factor of at least 50.
   Examples of cysE variants encoding CysE enzymes having reduced feedback inhibition and preferred embodiments in the context of this invention are:
      cysE mutated: Mutation in the sequence region from position 97 up to and including position 273.
      cysE deleted: Deletion in the sequence region from position 248 up to and including position 259.
         Specific mutations that are preferred embodiments in the context of this invention are:
      cysEII: G238S, mutation of glycine at position 238 to serine.
      cysEIII: G165D, mutation of glycine at position 165 to aspartic acid.
      cysEIV: A237V, mutation of alanine at position 237 to valine and G238S, mutation of glycine at position 238 to serine (double mutant).
      cysEV: A237V, mutation of alanine at position 237 to valine and G238S, mutation of glycine at position 238 to serine and M256I, mutation of methionine at position 256 to isoleucine (triple mutant).
      cysEVI: G238S, mutation of glycine at position 238 to serine and M256I, mutation of methionine at position 256 to isoleucine (double mutant).
      cysEVII: A237V, mutation of alanine at position 237 to valine.
      cysEVIII: M256I, mutation of methionine at position 256 to isoleucine and A237V, mutation of alanine at position 237 to valine (double mutant).
      cysEX: T167A, mutation of threonine at position 167 to alanine (used in the vector pCys, Example 1).
      cysEXI: T167A, mutation of threonine at position 167 to alanine and G245S, mutation of glycine at position 245 to serine (double mutant).
      cysEXIl: K97Q, mutation of lysine at position 97 to glutamine and G238S, mutation of glycine at position 238 to serine and F267L, mutation of phenylalanine at position 267 to leucine (triple mutant).
      cysEXIII: V164A, mutation of valine at position 164 to alanine and F267L, mutation of phenylalanine at position 267 to leucine (double mutant).
      cysEXIV: T167A, mutation of threonine at position 167 to alanine and M256Stop, mutation of methionine at position 256 to Stop (double mutant).
      cysEXVI: D250G, mutation of aspartic acid at position 250 to glycine.
      cysEXVII: G165D, mutation of glycine at position 165 to aspartic acid and T167A, mutation of threonine at position 167 to alanine (double mutant).
      cysEXXIII: T167A, mutation of threonine at position 167 to alanine and A237V, mutation of alanine at position 237 to valine and G238S, mutation of glycine at position 238 to serine (triple mutant).
      cysE M256X: Mutation of methionine at position 256 of the WT CysE enzyme to X, where X can be any natural amino acid except methionine.
      cysE Arabidopsis thaliana (plant): A. thaliana contains three cysE genes, two of which are feedback-resistant to cysteine.
      cysE M256I: Mutation of methionine at position 256 of the WT CysE enzyme to isoleucine.
   and/or
e) The production strain comprises, compared to the parent strain (host strain), at least one highly expressed cds e (overexpression) or an additional cds e encoding a cysteine exporter (-efflux protein), which leads to increased expression of cysteine exporters, such that in the cell of the production strain, cysteine export out of the cell is increased by a factor of at least two relative to the corresponding parent strain (host strain), where cysteine export can be determined by photometric measurement of the extracellular cysteine content according to Gaitonde, Biochem. J. (1967) 104: 627-633 (comprising cysteine, cystine and the adduct (R)-2-methylthiazolidine-2,4-dicarboxylic acid formed from cysteine and pyruvate), as described for example in U.S. Pat. No. 5,972,663 B.
   The increased expression of cysteine exporters results in cysteine export out of the cell being increased relative to the parent strain (host strain) preferably by a factor of at least 5, particularly preferably by a factor of at least 10, and especially preferably by a factor of at least 20.
   The cds encoding the cysteine exporter is preferably one or more sequences selected from the group consisting of ydeD, yfiK, cydDC, bcr and emrAB of E. coli or an at least 80%, particularly preferably at least 90% and especially preferably at least 95% identical sequence.

The cds that bring about hypotaurine biosynthesis and the cds that bring about the deregulated cysteine biosynthesis pathway are located extrachromosomally on vectors in the microorganism strain, either combined on one vector or divided over multiple vectors.

In a preferred embodiment, the process for producing hypotaurine is characterized in that the cds encoding CDO (a) and the cds encoding CSAD (b) in the expression vector of the microbial production strain are present with the cds encoding SerA having reduced feedback inhibition by serine (c) in a tricistronic expression unit which is particularly preferably functionally linked to the serA promoter, which especially preferably has the sequence specified in SEQ ID NO: 7. This means that the expression of the three aforementioned cds is regulated by only one promoter (i.e., there is a tricistronic operon). The 3 cds are separate and follow one another, and each cds has its own RBS. They are expressed in a coordinated manner under the regulation of the same promoter, which is preferably the serA promoter, but the result is 3 separate proteins instead of fusion proteins.

Particularly preferably, the order in the operon is such that the promoter, preferably the serA promoter, is followed by the serA cds, then the CDO cds and finally the CSAD cds in the tricistronic operon. serA is preferably serA317. SerA317 is disclosed in Bell et al., Eur. J. Biochem. (2002) 269: 4176-4184, referred to therein as “NSD:317”, and encodes a serine feedback-resistant variant of 3-phosphoglycerate dehydrogenase. The tricistronic expression unit composed of serA, CDO and CSAD in the present invention allows the coupling of L-cysteine and hypotaurine metabolism through their coordinated expression under the control of the serA promoter.

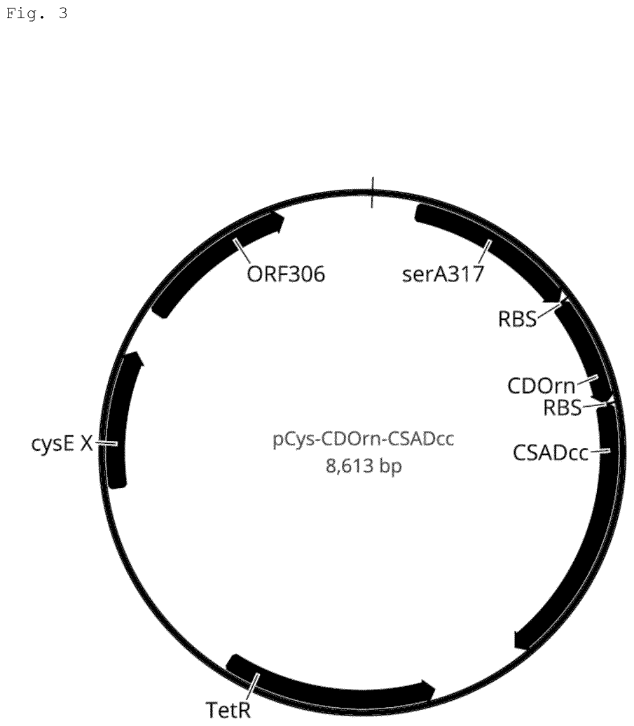

For example, in the expression vectors pCys-CDOrn-CSADhs (FIG. 2) and pCys-CDOrn-CSADcc (FIG. 3) disclosed in Example 1 of the present invention, the expression units serA317, CDOrn and CSADhs, and serA317, CDOrn and CSADcc, respectively form an artificial tricistronic operon, with expression taking place out under the control of the serA promoter. The serA promoter thus controls the expression of three cds in the gene constructs pCys-CDOrn-CSADhs and pCys-CDOrn-CSADcc, namely the expression of the cds encoding serA317 and the expression of the cds encoding the CDOrn enzyme, which was derived from the mRNA of the gene sequence available from Rattus norvegicus (rn), and, in the gene construct pCys-CDOrn-CSADhs, the expression of the cds encoding the CSADhs enzyme, which was derived from the mRNA of the gene sequence available from Homo sapiens (hs), and, in the gene construct pCys-CDOrn-CSADcc, the expression of the cds encoding the CSADcc enzyme, which was derived from the mRNA of the gene sequence available from Cyprinus carpio (cc).

A particularly preferred gene construct comprising a cds encoding SerA having reduced feedback inhibition by serine and functionally linked to the serA promoter (c), cysE having reduced feedback inhibition by cysteine and functionally linked to the cysE promoter (d) and a cysteine exporter functionally linked to the promoter of the E. coli GAPDH gene (e) (GAPDH: glyceraldehyde 3-phosphate dehydrogenase) is the vector pCys (FIG. 1) disclosed in WO 2021/259491 (Wacker) and described in Example 1. If pCys is transformed into a suitable host strain, what is obtained is a production strain which has a deregulated cysteine biosynthesis pathway and is therefore suitable for cysteine production (see for example WO 2021/259491, Wacker).

Particularly preferably, the process for producing hypotaurine is characterized in that the cds encoding CDO (a), CSAD (b), SerA having reduced feedback inhibition by serine (c), CysE having reduced feedback inhibition by cysteine (d) and encoding the cysteine exporter (e) are all present on a single expression vector. Especially preferably, the process for producing hypotaurine is characterized in that the expression vector of the microbial production strain is selected from the gene constructs pCys-CDOrn-CSADhs and pCys-CDOrn-CSADcc. Said gene constructs are disclosed in FIG. 2 and FIG. 3, respectively, of Example 1. In a specially preferred embodiment, the expression vector of the microbial production strain is the gene construct pCys-CDOrn-CSADcc.

The gene construct according to the invention thus comprises feedback-resistant alleles of the cysE gene and/or the serA gene and/or a gene for a cysteine exporter such as preferably the ydeD efflux gene and also genes encoding a CDO enzyme and a CSAD enzyme, the CDO gene and the CSAD gene, each with its own RBS, being linked to the cysE gene, the serA gene and/or the cysteine efflux gene, such as preferably the ydeD gene, in polycistronic units. Preference is given to a tricistronic operon, conceivable configurations including the following:
a) serA-CDO-CSAD
b) serA-CSAD-CDO
c) cysE-CDO-CSAD
d) cysE-CSAD-CDO
e) ydeD-CDO-CSAD
f) ydeD-CSAD-CDO.
a) and b) are preferably functionally linked to the serA promoter in the tricistronic operon, c) and d) are preferably functionally linked to the cysE promoter in the tricistronic operon, and e) and f) are preferably functionally linked to the promoter of the E. coli GAPDH gene in the tricistronic operon, though a) to f) can be functionally linked to any other promoter active in E. coli.

Preference is given to a gene construct comprising a tricistronic operon comprising
a) serA-CDO-CSAD or
b) serA-CSAD-CDO.

Particular preference is given to a gene construct comprising a tricistronic operon comprising
a) serA-CDO-CSAD.

As described in Example 1 of the present invention, the vector pCys (FIG. 1) described in WO 2021/259491 (Wacker) was extended by the genes for cysteine dioxygenase from Rattus norvegicus (CDOrn), SEQ ID NO: 1, encoding a protein having the amino acid sequence SEQ ID NO: 2, for L-cysteine sulfinic acid decarboxylase from Homo sapiens (CSADhs), SEQ ID NO: 3, nt 1 to nt 1509, encoding a protein having the amino acid sequence SEQ ID NO: 4, or for L-cysteine sulfinic acid decarboxylase from Cyprinus carpio (CSADcc), SEQ ID NO: 5, nt 1 to nt 1503, encoding a protein having the amino acid sequence SEQ ID NO: 6. The result was the vectors pCys-CDOrn-CSADhs (FIG. 2) and pCys-CDOrn-CSADcc (FIG. 3) for the production of hypotaurine. The gene constructs were produced according to the prior art and preferably with the aid of customary recombinant DNA techniques, as described in Example 1 and as are familiar to a person skilled in the art. In pCys-CDOrn-CSADhs, the CDOrn gene and the CSADhs gene are cloned in the form of an artificial tricistronic operon, each with its own ribosome binding site (RBS) but without its own promoter, after the serA317 gene present in pCys, such that their expression takes place under the control of the serA promoter. In pCys-CDOrn-CSADcc, the CDOrn gene and the CSADcc gene are cloned in the form of an artificial tricistronic operon, each with its own ribosome binding site (RBS) but without its own promoter, after the serA317 gene present in pCys, such that their expression takes place under the control of the serA promoter.

A production strain is produced in known fashion by transforming the gene construct according to the invention into the microorganism (host strain), preferably the gene construct pCys-CDOrn-CSADhs or the gene construct pCys-CDOrn-CSADcc, particularly preferably the gene construct pCys-CDOrn-CSADcc, into the host strain E. coli K12 W3110.

As disclosed in the examples of the present invention, the fermentation of production strains according to the invention, characterized by the previously unknown coordinated expression of the CDO gene and CSAD gene by a polycistronic, preferably tricistronic operon under the control of the serA promoter stemming from cysteine metabolism, allows surprisingly far higher production of hypotaurine compared to the prior art. Disclosed in the prior art, for example in WO 17/213142 A1 (Ajinomoto), are hypotaurine yields in E. coli of a maximum of 450 μM, corresponding to 49.1 mg/L hypotaurine with the molecular weight of hypotaurine being 109.2 g/mol. Honjoh et al. (2010), Amino Acids 38: 173-1183, describe only the intracellular production of hypotaurine in yeast with yields of 85.3 μmol/g dry biomass, corresponding to 9.3 mg/g hypotaurine with the molecular weight of hypotaurine being 109.2 g/mol. However, the growth medium had to receive the addition of L-cysteine as substrate of hypotaurine biosynthesis (see “Materials and Methods” in Honjoh et al.).

Furthermore, the prior art only describes production yields for taurine, the oxidation product of hypotaurine. For instance, US 2019/0062757 A1 (KnipBio) discloses yields of 419 ng/ml taurine (E. coli), whereas Joo et al. (2018), J. Agric. Food Chem. 66: 13454-13463, mentions taurine yields in C. glutamicum of a maximum of 0.5 g/L taurine.

Preferably, the process for fermentative production of hypotaurine is characterized in that the fermentation volume is at least 1 L, a production scale of at least 10 L being particularly preferred and that of at least 1000 L being especially preferred, and a fermentation volume of at least 10 000 L being specifically preferred.

The content of hypotaurine may be quantified from the culture broth. This is done by taking, for example, a 1 ml aliquot of the culture broth having a cell density OD₆₀₀/ml of at least 1.0/ml and incubating it at 80 °C for 5 min, followed by removing all solid constituents, for example by centrifuging at maximum speed in a benchtop centrifuge for five minutes, and quantifying the supernatant by HPLC calibrated for hypotaurine and taurine, as described for hypotaurine and taurine inter alia in Example 3.

In the process according to the invention, the yield of the product hypotaurine at the end of fermentation after a fermentation time of preferably a maximum of 65 h is preferably at least 10 g/L, particularly preferably at least 20 g/L and especially preferably at least 50 g/L, far greater than is known in the prior art (e.g., 49.1 mg/L hypotaurine in WO 17/213142 A1). The process according to the invention thus allows the fermentative production of hypotaurine in previously unknown yields for applications in the food sector, the feed sector and also the pharmaceutical sector.

The fermentative process according to the invention is additionally distinguished in that it allows the production of hypotaurine in a high molar excess relative to the by-product taurine. The molar excess is defined as the quotient of the molar concentration of hypotaurine in the fermentation supernatant at the end of fermentation divided by the molar concentration of taurine in the fermentation supernatant at the same time. As disclosed in Example 4, the molar excess of hypotaurine:taurine is at least 3:1, preferably at least 5:1, particularly preferably at least 10:1 and especially preferably at least 20:1.

Media for growth of the production strain in a shake flask and by fermentation are familiar to a person skilled in the art from practising microbial cultivation. They typically consist of a carbon source, a nitrogen source, and additives such as vitamins, salts and trace elements, and a sulfur source which optimizes cell growth and hypotaurine production.

Carbon sources are those that can be used by the production strain for formation of hypotaurine product. These include all forms of monosaccharides, comprising C6 sugars (hexoses) such as glucose, mannose, fructose or galactose and C5 sugars (pentoses) such as xylose, arabinose or ribose, and all conceivable di- and polysaccharides formed therefrom, such as sucrose, lactose, maltose, maltodextrin, starch, and the monomers or oligomers released (enzymatically or chemically) therefrom by hydrolysis. Other usable carbon sources other than sugars or carbohydrates are acetic acid (or acetate salts derived therefrom), ethanol, glycerol, citric acid (and salts thereof) or pyruvate (and salts thereof). However, gaseous carbon sources such as carbon dioxide or carbon monoxide are also conceivable. Preferred carbon sources for growth of the production strain are glucose, fructose, sucrose, mannose, xylose and arabinose, among which glucose and sucrose are particularly preferred and glucose is especially preferred.

Nitrogen sources are those that can be used by the production strain for formation of biomass. Examples of what may be used for this purpose include:
ammonia, in gaseous form or in aqueous solution as NH₄OH, or else salts thereof such as ammonium sulfate, ammonium chloride, ammonium phosphate, ammonium acetate or ammonium nitrate, and/or
the known nitrate salts such as KNO₃, NaNO₃, ammonium nitrate, Ca(NO₃)₂, Mg(NO₃)₂ and other nitrogen sources such as urea, and/or
complex mixtures of amino acids such as yeast extract, proteose peptone, malt extract, soy peptone, casamino acids, corn steep liquor (in liquid form or else in dried form as so-called CSD), and/or
NZ Amine, and/or
yeast nitrogen base.

The metered addition of a sulfur source, either as a one-time addition in batch form or as a continuous feed, is required for the efficient production of hypotaurine.

Continuous metered addition may be effected as a pure feed solution or else in a mixture with a further feed component such as glucose. Preferably, the process is characterized in that it is carried out in the presence of at least one compound selected from the group consisting of a salt of sulfates, sulfites, dithionites, thiosulfates and sulfides, the use of the respective acids also being conceivable at a given stability. Preferred sulfur sources are salts of sulfates, sulfites, thiosulfates and sulfides, among which particular preference is given to salts of sulfates and thiosulfates. Especially preferably, the process is characterized in that it is carried out in the presence of thiosulfate salts. Particularly preferred as thiosulfate salt is a compound selected from the group consisting of sodium thiosulfate, ammonium thiosulfate and mixtures thereof.

Growth (cultivation of the microorganism cells) may be carried out in so-called batch mode, i.e., obtaining biomass by inoculation of the growth medium (culture medium) with a starter culture of the production strain (microorganism cells bearing one or more gene constructs) and then cell growth without further feeding of nutrient sources. Growth may also be carried out in so-called fed-batch mode (also called growth in feed mode), i.e., a process for obtaining biomass, in which an initial phase of growth in batch mode is followed by additional feeding of nutrient sources (feed). The feed may consist of the carbon source, the nitrogen source, the sulfur source, one or more vitamins or trace elements important for production, or a combination of the foregoing. The feed components may be metered in together as a mixture or else separately in individual feed sections. In addition, other media constituents, and additives which specifically increase hypotaurine production, may also be added to the feed. The feed may be supplied continuously or in portions (discontinuously), or else in a combination of continuous and discontinuous feed. Preferably, the process according to the invention for fermentative production of hypotaurine using a microbial production strain is characterized in that the fermentative process is a fed-batch mode process.

Preferred carbon sources in the feed are glucose, sucrose, and glucose- or sucrose-containing vegetable hydrolysates, and mixtures of the preferred carbon sources in any mixing ratio. A particularly preferred carbon source in the feed is glucose.

Preferably, the carbon source is metered into the culture such that the content of carbon source in the fermenter during the production phase does not exceed 10 g/L. Preference is given to a maximum concentration of 2 g/L, particularly preferably 0.5 g/L, especially preferably 0.1 g/L.

Preferred nitrogen sources in the feed are ammonia, in gaseous form or in aqueous solution as NH₄OH, and its salts ammonium sulfate, ammonium phosphate, ammonium acetate and ammonium chloride, and additionally urea, KNO₃, NaNO₃ and ammonium nitrate, yeast extract, proteose peptone, malt extract, soy peptone, casamino acids, corn steep liquor and also NZ Amine and yeast nitrogen base, among which particular preference is given to ammonia or ammonium salts, yeast extract, soy peptone or corn steep liquor (in liquid form or in dried form).

Preferred sulfur sources in the feed are salts of sulfates, sulfites, thiosulfates and sulfides, among which salts of sulfates and thiosulfates are particularly preferred and thiosulfate salts, such as sodium thiosulfate and ammonium thiosulfate, are especially preferred.

Further media supplements that may be added are salts of the elements phosphorus, chlorine, sodium, magnesium, nitrogen, potassium, calcium and iron, and in trace amounts (i.e., in μM concentrations), salts of the elements molybdenum, boron, cobalt, manganese, zinc, copper and nickel. Furthermore, organic acids (e.g., acetate, citrate), amino acids (e.g., isoleucine) and vitamins (e.g., vitamin BI, vitamin B6) may be added to the medium.

Growth is carried out under pH and temperature conditions which promote growth and hypotaurine production of the production strain. The pH range preferably ranges from pH 5 to pH 9. Particular preference is given to a pH range from pH 5.5 to pH 8. Especial preference is given to a pH range from pH 6.0 to pH 7.5.

The preferred temperature range for growth of the production strain is 20 °C to 40 °C. The temperature range is particularly preferably from 25 °C to 37 °C and especially preferably from 28 °C to 34 °C.

Growth of the production strain may optionally take place without supply of oxygen (anaerobic cultivation) or else with supply of oxygen (aerobic cultivation). Preference is given to aerobic cultivation with oxygen.

In the case of aerobic cultivation of the strain according to the invention for hypotaurine production, a saturation of preferably at least 10% (v/v), particularly preferably of at least 20% (v/v) and especially preferably of at least 30% (v/v) is set for the oxygen content. In accordance with the prior art, the oxygen saturation in the culture is regulated automatically via a combination of gas supply and stirring speed. The supply of oxygen may be ensured by introduction of compressed air or pure oxygen. Preference is given to aerobic cultivation by introduction of compressed air. The compressed air supply in aerobic cultivation preferably ranges from 0.05 vvm to 10 vvm (vvm: introduction of compressed air into the fermentation batch specified in liters of compressed air per liter of fermentation volume per minute). An introduction of compressed air of 0.2 vvm to 8 vvm is particularly preferred, that of 0.4 to 6 vvm is especially preferred, and that of 0.8 to 5 vvm is specifically preferred.

The maximum stirring speed is preferably 2500 rpm, particularly preferably a maximum of 2000 rpm and especially preferably a maximum of 1800 rpm.

The cultivation time is preferably between 10 h and 200 h. Particular preference is given to a cultivation time of 20 h to 120 h. Especial preference is given to a cultivation time of 30 h to 100 h.

In the process according to the invention for fermentative production of hypotaurine, the microbial production strain according to the claims is cultivated in step i and the fermentation supernatant is isolated in step ii. This means that fermentation batches obtained by the process described contain hypotaurine that has accumulated extracellularly in the fermentation supernatant. Hypotaurine may be used further directly or may be isolated from the fermentation medium. Various analytical methods for identifying, quantifying and determining the degree of purity of hypotaurine are available, including spectrophotometry, NMR, gas chromatography, HPLC, mass spectroscopy, gravimetry or a combination of these analytical methods.

The figures show the plasmids used in the examples.

FIG. 1: pCys.

FIG. 2: pCys-CDOrn-CSADhs.

FIG. 3: pCys-CDOrn-CSADcc.

### ABBREVIATIONS USED IN THE FIGURES

TetR: Gene conferring resistance to tetracycline
P15A ORI: Origin of replication
serA317: serA (3-phosphoglycerate dehydrogenase gene encoding amino acids 1 to 317) cds
cysE X: cysE (serine O-acetyltransferase gene, feedback resistant) cds
ORF306: ydeD (cysteine efflux gene) cds
ScaI: Cleavage site for the restriction enzyme ScaI
PpuMI: Cleavage site for the restriction enzyme PpuMI
CDOrn: CDO (cysteine dioxygenase) R. norvegicus cds
CSADhs: CSAD (cysteine sulfinic acid decarboxylase) H. sapiens cds
CSADcc: CSAD (cysteine sulfinic acid decarboxylase) C. carpio cds
RBS: Ribosome binding site

The following examples serve to further elucidate the invention:

### Example 1: Production of pCys-CDOrn-CSADhs and pCys-CDOrn-CSADcc

The vectors pCys-CDOrn-CSADhs and pCys-CDOrn-CSADcc are derived from the vector pCys (FIG. 1).

pCys: pCys is disclosed in WO 2021/259491 (Wacker) and is a derivative of the plasmid pACYC184-cysEX-GAPDH-ORF306. The plasmid pACYC184-cysEX-GAPDH-ORF306 contains not only the origin of replication and a tetracycline resistance gene (parent vector pACYC184), but also the cysEX allele (synonym: cysE allele), which encodes a serine O-acetyltransferase having reduced feedback inhibition by cysteine and the expression of which is controlled by the cysE promoter, and also the efflux gene ydeD (ORF306), the expression of which is controlled by the constitutive GAPDH promoter stemming from the E. coli GAPDH gene.

Furthermore, pCys additionally contains the serA317 gene fragment, which is cloned after the ydeD (ORF306) efflux gene and which encodes the N-terminal 317 amino acids of the SerA protein (total length: 410 amino acids). The E. coli serA gene is disclosed in the “GenBank” gene database with the gene ID 945258. serA317 is disclosed in Bell et al., Eur. J. Biochem. (2002) 269: 4176-4184, referred to therein as “NSD:317”, and encodes a serine feedback-resistant variant of 3-phosphoglycerate dehydrogenase. The expression of serA317 is controlled by the serA promoter. The serA promoter according to SEQ ID NO: 7 is disclosed in Rex et al. (1991), J. Bacteriol. 173: 5944-5953, FIG. 2, nt 1-nt 457.

For cloning purposes, plasmid DNA of the vector pCys was cut with ScaI and PpuMI, and the 6.1 kb vector fragment, referred to hereinafter as pCys-ScaI/PpuMI, was isolated by preparative agarose gel electrophoresis (QIAquick® Gel Extraction Kit, Qiagen).

CDOrn: The amino acid sequence of cysteine dioxygenase from Rattus norvegicus, derived from the mRNA of the gene, is disclosed in the NCBI (National Center for Biotechnology Information) database under the sequence ID: AAH70509.1. The amino acid sequence was used to derive a DNA sequence codon-optimized for expression in E. coli (publicly available Eurofins Genomics GENEius software), and said DNA sequence was synthetically produced (Eurofins Genomics). The CDOrn DNA sequence is disclosed in SEQ ID NO: 1 and encodes a protein having the amino acid sequence from SEQ ID NO: 2. The CDOrn DNA was synthetically produced (Eurofins Genomics).

### CSADhs-rrnB:

CSADhs: The amino acid sequence of cysteine sulfinic acid decarboxylase (CSADhs) from Homo sapiens is disclosed in the NCBI (National Center for Biotechnology Information) database under the sequence ID: XP_016861786.1. CSADhs is deposited there as “acidic amino acid decarboxylase GADL1 isoform X1”. The amino acid sequence was used to derive a DNA sequence codon-optimized for expression in E. coli (publicly available Eurofins Genomics GENEius software). This DNA sequence, designated CSADhs cds, is disclosed in SEQ ID NO: 3, nt 1 to 1509 and encodes a protein having the amino acid sequence from SEQ ID NO: 4. The DNA sequence of the E. coli rrnB terminator (SEQ ID NO: 3, nt 1510 to 1842) was linked to nt 1509. The DNA sequence of the rrnB terminator is disclosed in Orosz et al., Eur. J. Biochem. (1991) 201: 653-659. The DNA disclosed in SEQ ID NO: 3, consisting of the CSADhs cds and the rrnB terminator, was synthetically produced (Eurofins Genomics) and designated CSADhs-rrnB.

CSADcc-rrnB: The cDNA gene (cDNA: complementary DNA, isolated from mRNA by reverse transcription) of cysteine sulfinic acid decarboxylase (CSAD) from Cyprinus carpio (carp) was isolated by Honjoh et al. (2010), Amino Acids 38: 1173-183, and the DNA sequence disclosed in the NCBI database under the GenBank sequence ID: AB220585.1 (cds: nt 82-1584). The amino acid sequence was used to derive a DNA sequence codon-optimized for expression in E. coli (publicly available Eurofins Genomics GENEius software). This DNA sequence, designated CSADcc cds, is disclosed in SEQ ID NO: 5, nt 1 to 1503 and encodes a protein having the amino acid sequence from SEQ ID NO: 6. The 3′ end of the CSADcc cds was linked to the DNA sequence of the E. coli rrnB terminator (SEQ ID NO:5, nt 1504 to 1834). The DNA sequence of the rrnB terminator is disclosed in Orosz et al. (1991); see above. The CSADcc-rrnB DNA, consisting of the CSADcc cds and the rrnB terminator, was synthetically produced (Eurofins Genomics).

### Primers Used:

cdorn-1f (SEQ ID NO: 8)
csadhs-2r (SEQ ID NO: 9)
csadhs-3f (SEQ ID NO: 10)
glf-2r (SEQ ID NO: 11):
cdorn-3r (SEQ ID NO: 12)
csadcc-1f (SEQ ID NO: 13)
   PCR products:

Table 1 summarizes the PCR products used for cloning, in respect of their designation and their size, and the synthetic DNA (so-called template DNA) and primers used. The PCR products PCR1 to PCR4 were produced from the respective template DNA by PCR (“Phusion™ High-Fidelity” DNA Polymerase, Thermo Scientific™) using the appropriate primers and isolated by agarose gel electrophoresis (QIAquick® Gel Extraction Kit, Qiagen).

TABLE 1
PCR products for production of gene constructs

| PCR | PCR | | | |
|---|---|---|---|---|
| product | product size | Template DNA | Forward primer | Reverse primer |
| PCR1 | 0.6 kb | Synthetic CDOrn | cdorn-1f | csadhs-2r |
| | | DNA | SEQ ID NO: 8 | SEQ ID NO: 9 |
| PCR2 | 1.8 kb | Synthetic | csadhs-3f | glf-2r |
| | | CSADhs-rrnB | SEQ ID NO: 10 | SEQ ID NO: 11 |
| | | DNA | | |
| PCR3 | 0.6 kb | Synthetic CDOrn | cdorn-1f | cdorn-3r |
| | | DNA | SEQ ID NO: 8 | SEQ ID NO: 12 |
| PCR4 | 1.9 kb | Synthetic | csadcc-1f | glf-2r |
| | | CSADcc-rrnB | SEQ ID NO: 13 | SEQ ID NO: 11 |
| | | DNA | | |

Production of the Vector pCys-CDOrn-CSADhs:

The vector pCys-CDOrn-CSADhs was produced by using the NEBuilder® cloning kit (NEB New England Biolabs). The 6.1 kb pCys-ScaI/PpuMI vector fragment was ligated together with PCR1 and PCR2, following the manufacturer's instructions regarding the amounts of the individual DNA fragments used in the ligation mix. Thereafter, the ligation mix was transformed into E. coli NEB® 10-beta (NEB New England Biolabs) according to the manufacturer's instructions. Clones from the transformation were selected on LBtet plates. LBtet plates contained 10 g/L tryptone (GIBCO™), 5 g/L yeast extract (BD Biosciences), 5 g/L NaCl, 15 g/L agar and 15 mg/L tetracycline (Sigma-Aldrich). From clones of the transformation, the plasmid DNA was isolated (Qiagen Kit) and a correct clone was selected. The plasmid DNA of the clone was designated vector pCys-CDOrn-CSADhs (FIG. 2).

Production of the Vector pCys-CDOrn-CSADcc:

The vector pCys-CDOrn-CSADcc was produced by using the NEBuilder® cloning kit (NEB New England Biolabs). The 6.1 kb pCys-ScaI/PpuMI vector fragment was ligated together with PCR3 and PCR4, following the manufacturer's instructions regarding the amounts of the individual DNA fragments used in the ligation mix. Thereafter, the ligation mix was transformed into E. coli NEB® 10-beta (NEB New England Biolabs) according to the manufacturer's instructions. Clones from the transformation were selected on LBtet plates. From clones of the transformation, the plasmid DNA was isolated (Qiagen Kit) and a correct clone was selected. The plasmid DNA of the clone was designated vector pCys-CDOrn-CSADcc (FIG. 3).

### Example 2: Production of Production Strains

The parent strain (host strain) for production of production strains was the microorganism strain Escherichia coli K12 W3110 (commercially available under the strain number DSM 5911 from the DSMZ-German Collection of Microorganisms and Cell Cultures GmbH). E. coli W3110 was transformed in known fashion with the vector pCys-CDOrn-CSADhs or the vector pCys-CDOrn-CSADcc and transformants were selected on LBtet plates. One transformant each was selected as production strain. The production strains were designated E. coli W3110×pCys-CDOrn-CSADhs and E. coli W3110×pCys-CDOrn-CSADcc and were used for production of hypotaurine.

### Example 3: Production of Hypotaurine in Shake Flasks

Preculture: A preculture of the production strain E. coli W3110×pCys-CDOrn-CSADhs and a preculture of the production strain E. coli W3110×pCys-CDOrn-CSADcc were prepared in LBtet medium (10 g/L tryptone, 5 g/L yeast extract, 5 g/L NaCl, 15 mg/L tetracycline) (growth at 37 °C and 120 rpm overnight).

Main culture: 0.5 ml of the respective preculture was transferred to a 300 ml Erlenmeyer flask (baffled) containing 30 ml of SM1 medium supplemented with 15 g/L glucose, 2 g/L Na₂S₂O₃×5 H₂O, 0.1 g/L L-isoleucine, 0.1 g/L D,L-methionine, 0.1 g/L L-threonine, 5 mg/L vitamin B1 and 15 mg/L tetracycline.

Composition of SM1 medium: 12 g/L K2HPO₄, 3 g/L KH₂PO₄, 5 g/L NH₄ sulfate, 0.3 g/L MgSO₄×7 H₂O, 0.015 g/L CaCl₂)×2H₂O, 0.002 g/L FeSO₄×7 H₂O, 1 g/L Nas citrate×2H₂O, 0.1 g/L NaCl; 1 mi/L trace element solution.

Composition of the trace element solution: 0.15 g/L Na₂MoO₄×2 H₂O, 2.5 g/L H₃BO₃, 0.7 g/L COCl₂×6 H₂O, 0.25 g/L CuSO₄×5 H₂O, 1.6 g/L MnCl₂×4 H₂O, 0.3 g/L ZnSO₄×7 H₂O.

The main cultures were incubated at 30 °C and 140 rpm for 24 h in an incubator shaker (Infors). After 24 h, 1 ml samples were taken and the cell density OD600/ml (optical density of the main culture, measured photometrically at 600 nm) was measured using a Genesys™ 10S UV/visible spectrophotometer from Thermo Scientific™, and the content of hypotaurine and taurine was determined by HPLC. For the strain E. coli W3110×pCys-CDOrn-CSADhs, the content of hypotaurine was 54.9 mg/L. The content of taurine was 44.6 mg/L. For the strain E. coli W3110×pCys-CDOrn-CSADcc, the content of hypotaurine was 158.8 mg/L. The content of taurine was 8.7 mg/L.

Sample preparation for quantification of hypotaurine and taurine by HPLC: 1 ml of culture broth from the shake flask growth or the fermentation was incubated at 80 °C for 5 min and then centrifuged at 13 000 rpm for 5 min (Heraeus™ Fresco™ 21 centrifuge). The cell culture supernatant was isolated. Cell culture supernatants from the shake flask growth were analyzed directly by HPLC for the content of hypotaurine and taurine. Cell culture supernatants from the fermentation were diluted 10-fold in H₂O and analyzed by HPLC for the content of hypotaurine and taurine.

HPLC analysis of L-cysteine sulfinic acid, hypotaurine and taurine: For quantitative determination of the compounds quantitatively analyzed in the examples, and for L-cysteine sulfinic acid for detection of CDO enzyme activity, an HPLC method calibrated respectively for L-cysteine sulfinic acid, hypotaurine and taurine was employed; the reference substances used for calibration were commercially available (Sigma-Aldrich). An Agilent 1260 Infinity II HPLC system was used, which was equipped with a unit from the same manufacturer for pre-column derivatization with o-phthaldialdehyde (OPA derivatization) as is known from the analysis of amino acids. For detection of the OPA-derivatized products of hypotaurine and taurine, the HPLC system was equipped with a fluorescence detector. The detector was set to an excitation wavelength of 330 nm and an emission wavelength of 450 nm. Also used were an Accucore™ aQ column from Thermo Scientific™, length 100 mm, internal diameter 4.6 mm, particle size 2.6 μm, thermally equilibrated at 40 °C in a column oven.

Eluent A: 25 mM Na phosphate, pH 6.0. Eluent B: methanol. The separation was carried out in gradient mode: 10% eluent B to 60% eluent B over 0-25 min, followed by 60% eluent B to 100% eluent B over 2 min, followed by 100% eluent B for a further 2 min, at a flow rate of 0.5 ml/min. Retention time of L-cysteine sulfinic acid: 4.1 min. Retention time of taurine: 14.8 min. Retention time of hypotaurine: 15.7 min.

### Example 4: Production of Hypotaurine by Fermentation

### Preculture 1:

The production strains E. coli W3110×pCys-CDOrn-CSADhs and E. coli W3110×pCys-CDOrn-CSADcc were each inoculated in 20 ml of LBtet medium in a 100 ml Erlenmeyer flask and incubated on a shaker (150 rpm, 30 °C) for 7 h.

### Preculture 2:

Thereafter, the precultures 1 were each entirely transferred to 100 ml of SM1 medium supplemented with 5 g/L glucose, 5 mg/L vitamin B1 and 15 mg/L tetracycline (for the composition of SM1 medium, see Example 3). The cultures were each shaken in an Erlenmeyer flask (1 L volume) at 30 °C for 17 h at 150 rpm (Infors incubator shaker). Following this incubation, the cell densities OD₆₀₀/ml were each between 3 and 5.

### Main Culture:

The fermentations were carried out in a Biostat B fermenter (2 I working volume) from Sartorius BBI Systems GmbH.

The growth medium (900 ml) contained 15 g/L glucose, 10 g/L tryptone (Difco), 5 g/L yeast extract (Difco), 2.4 g/L (NH₄)₂SO₄, 5 g/L KH₂PO₄, 0.25 g/L NaCl, 0.6 g/L MgSO₄×7 H₂O, 0.03 g/L CaCl₂)×2H₂O, 0.15 g/L FeSO₄×7 H₂O, 1 g/L Nas citrate×2H₂O and 1 ml of trace element solution (see Example 3), 0.9 g/L L-isoleucine (Sigma-Aldrich), 0.6 g/L D,L-methionine (Sigma-Aldrich), 0.018 g/L vitamin BI (Sigma-Aldrich), 0.09 g/L pyridoxine×HCl (vitamin B6, Sigma-Aldrich) and 15 mg/L tetracycline.

The pH in the fermenter was initially adjusted to 7.0 by pumping in a 25% NH₄OH solution. During the fermentation, the pH was maintained at a value of 7.0 by automatic correction with 25% NH₄OH or 4 M H₃PO₄. Foam control was achieved by automatic metered addition of 4% v/v Struktol J673 in H₂O (Schill & Seilacher).

For inoculation, 100 ml of preculture 2 were pumped into the fermenter vessel in each case. The initial volume was therefore 1 L. The cultures were initially stirred at 400 rpm and aerated with compressed air sterilized via a sterile filter at an aeration rate of 2 vvm (vvm: introduction of compressed air into the fermentation batch specified in liters of compressed air per liter of fermentation volume per minute). Under these starting conditions, the oxygen probe was calibrated to 100% saturation prior to inoculation.

The target value for the O₂ saturation during the fermentation was set to 30%. After the O₂ saturation had fallen below the target value, a regulation cascade was started in order to bring the O₂ saturation back up to the target value. This involved first increasing the gas supply continuously (to a maximum of 5 vvm) and then increasing the stirring speed continuously (to a maximum of 1500 rpm).

The fermentation was carried out at a temperature of 30 °C. After a fermentation time of 2 h, a sulfur source in the form of a sterile 60% (w/v) stock solution of sodium thiosulfate×5H₂O was fed in at a rate of 1.5 ml per hour.

Once the glucose content in the fermenter had fallen from an initial 15 g/L to approx. 2 g/L, a 56% (w/w) glucose solution was continuously metered in. The feeding rate was adjusted such that the glucose concentration in the fermenter no longer exceeded 2 g/L from then on. Glucose was determined using a glucose analyzer from YSI (Yellow Springs, Ohio, USA).

The fermentation time was 65 h. 22 h, 40 h and 65 h after the start of fermentation, samples were taken from the fermentation batch, followed by determination of the cell density OD₆₀₀/ml and determination of the content of hypotaurin and taurine in the culture supernatant by HPLC. The results are summarized in Table 2 and Table 3.

For the strain W3110×pCys-CDOrn-CSADhs, after a fermentation time of 65 h, the hypotaurine yield was 20.8 g/L (190.5 mM with the molecular weight of hypotaurine being 109.2 g/mol) and the taurine yield was 7.4 g/L (59.1 mM with the molecular weight of taurine being 125.2 g/mol). The molar ratio of hypotaurine:taurine was 3.2:1.

For the strain W3110×pCys-CDOrn-CSADcc, after a fermentation time of 65 h, the hypotaurine yield was 62.5 g/L (572.2 mM with the molecular weight of hypotaurine being 109.2 g/mol) and the taurine yield was 1.2 g/L (9.6 mM with the molecular weight of taurine being 125.2 g/mol). The molar ratio of hypotaurine:taurine was 59.6:1.

TABLE 2
Time course of cell density and content of hypotaurine
and taurine in the fermentation of the production
strain W3110 × pCys-CDOrn-CSADhs

| Fermen- | | | |
|---|---|---|---|
| tation time | Cell density | Hypotaurine | Taurine |
| [h] | OD₆₀₀/ml | [g/L] | [g/L] |
| 22 | 74.0 | 10.5 | 3.3 |
| 40 | 147.1 | 18.0 | 6.9 |
| 65 | 166.3 | 20.8 | 7.4 |

TABLE 3
Time course of cell density and content of hypotaurine
and taurine in the fermentation of the production
strain W3110 × pCys-CDOrn-CSADcc

| Fermen- | | | |
|---|---|---|---|
| tation time | Cell density | Hypotaurine | Taurine |
| [h] | OD₆₀₀/ml | [g/L] | [g/L] |
| 22 | 78.2 | 11.5 | 0.0 |
| 40 | 159.2 | 40.4 | 0.0 |
| 65 | 179.4 | 62.5 | 1.2 |

### Example 5: Extraction of Hypotaurine and Taurine from Fermenter Cells

Use was made of the fermentation batch of the production strain E. coli W3110×pCys-CDOrn-CSADcc from Example 4 having a hypotaurine content of 62.5 g/L and taurine content of 1.2 g/L (Table 3). 2×1 ml of the fermenter broth (2 ml total volume) were centrifuged at 13 000 rpm for 5 min (Heraeus™ Fresco™ 21 centrifuge) and the supernatant was discarded. The cell pellets were resuspended in 1 ml of H₂O each and centrifuged at 13 000 rpm for 5 min, and the supernatant was discarded. The cell pellets were suspended in 1 ml of H₂O each, corresponding to 2 ml total volume and corresponding to the volume of fermenter broth as used above. The cell suspension was used to prepare a cell extract. This was accomplished by using the FastPrep-24TM 5G cell homogenizer from MP Biomedicals. The cell pellets suspended in 1 ml of H₂O each were disrupted in manufacturer-assembled 1.5 ml tubes containing glass beads (“Lysing Matrix B”) (3×20 sec at a shaking frequency of 6000 rpm with a pause between the intervals of 30 sec in each case). The cell homogenates obtained were combined and centrifuged at 13 000 rpm for 5 min in order to prepare a cell extract. The cell extract were analyzed by HPLC for the content of hypotaurine und taurine. Neither hypotaurine nor taurine was detected at a detection limit in the HPLC analysis of 1 mg/L for hypotaurine/taurine.

1-12. (canceled)

13. A process for fermentative production of hypotaurine, characterized in that
i) a microbial production strain is cultivated, said microbial production strain being distinguished in that it
1. contains at least one expression vector comprising a coding sequence (cds) a and a cds b and at least one of the cds selected from the group consisting of c, d and e,
a) a being a cds encoding a cysteine dioxygenase (CDO) belonging to enzyme class EC 1.13.11.20 and
b) b being a cds encoding a cysteine sulfinic acid decarboxylase (CSAD) belonging to enzyme class EC 4.1.1.29 and
c) c being a cds encoding a 3-phosphoglycerate dehydrogenase (SerA) in which feedback inhibition by serine is reduced by a factor of at least two relative to the corresponding wild-type enzyme and
d) d being a cds encoding a serine O-acetyltransferase (CysE) in which feedback inhibition by cysteine is reduced by a factor of at least two relative to the corresponding wild-type enzyme and
e) e being a cds encoding a cysteine exporter, the cds encoding the cysteine exporter being one or more sequences selected from the group consisting of ydeD, yfiK, cydDC, bcr and emrAB of E. coli or an at least 80% identical sequence,
2. the expression of the cds a and b in the expression vector is coordinated with the expression of at least one cds selected from the group consisting of c, d and e in a polycistronic expression unit,
3. hypotaurine is secreted by the production strain into the fermentation supernatant, and
ii) the fermentation supernatant is isolated,
the hypotaurine content in the fermentation supernatant being at least 10 g/L and the molar fraction of hypotaurine:taurine in the fermentation supernatant being at least 3:1.

14. The process as claimed in claim 13, wherein the host strain used for producing the microbial production strain is a microorganism of the species Escherichia coli.

15. The process as claimed in claim 13, wherein the CDO is the CDO from Rattus norvegicus (CDOrn) having the sequence specified in SEQ ID NO: 2 or an at least 80% identical enzyme having CDO activity.

16. The process as claimed in claim 13, wherein the CSAD is the CSAD from Cyprinus carpio (CSADcc) having the sequence specified in SEQ ID NO: 6 or an at least 80% identical enzyme having CSAD activity.

17. The process as claimed in claim 13, wherein the CSAD is the CSAD from Homo sapiens (CSADhs) having the sequence specified in SEQ ID NO: 4 or an at least 80% identical enzyme having CSAD activity.

18. The process as claimed in claim 13, wherein the cds encoding CDO (a) and the cds encoding CSAD (b) in the expression vector of the microbial production strain are present with the cds encoding SerA having reduced feedback inhibition by serine (c) in a tricistronic expression unit which is functionally linked to the serA promoter.

19. The process as claimed in claim 13, wherein the cds encoding CDO (a), CSAD (b), SerA having reduced feedback inhibition by serine (c), CysE having reduced feedback inhibition by cysteine (d) and encoding the cysteine exporter (e) are all present on a single expression vector.

20. The process as claimed in claim 13, wherein the expression vector of the microbial production strain is selected from the gene constructs pCys-CDOrn-CSADhs and pCys-CDOrn-CSADcc.

21. The process as claimed in claim 13, wherein the fermentation volume is at least 1 L.

22. The process as claimed in claim 13, wherein the process is carried out in the presence of thiosulfate salts.

23. The process as claimed in claim 13, wherein the fermentative process is a fed-batch mode process.

24. The process as claimed in claim 13, wherein the hypotaurine content in the fermentation supernatant based on the hypotaurine content in the fermentation batch is above 70%.


SEQUENCE LISTING (13 sequences)

SEQ ID NO: 1 - DNA, 603 bp, synthetic construct
  source 1..603: mol_type = other DNA
atggaacgcaccgaactgctgaaaccgcgtaccttggctgatctgattcggattctgcatgaactgtttgccggtgatgaggtcaatgttgaggaagtgcaagcagttctggaggcgtatgaaagcaatccggcagaatgggcgttatacgccaaatttgaccagtatcgctatacgcgtaatctggtagatcagggtaatggcaaattcaacctgatgatcttatgctggggagaaggtcatgggagtagcattcacgatcataccgatagccattgctttctgaaactcttgcaaggcaatctgaaagaaaccctctttgattggccggataagaaatccaacgaaatgatcaagaaatctgaacgtactcttcgcgaaaatcagtgtgcgtacatcaacgactccattggtttgcaccgcgtagagaacgtcagccataccgaaccagctgtgtcactgcacctttactctcctccgtttgacacgtgtcatgcctttgaccagcgtacaggccacaagaacaaagtgacgatgaccttccactcgaaattcgggattcgcacacccttcacgactagtggctcgttagagaacaactaa

SEQ ID NO: 2 - protein, 200 aa, Rattus norvegicus
MERTELLKPRTLADLIRILHELFAGDEVNVEEVQAVLEAYESNPAEWALYAKFDQYRYTRNLVDQGNGKFNLMILCWGEGHGSSIHDHTDSHCFLKLLQGNLKETLFDWPDKKSNEMIKKSERTLRENQCAYINDSIGLHRVENVSHTEPAVSLHLYSPPFDTCHAFDQRTGHKNKVTMTFHSKFGIRTPFTTSGSLENN

SEQ ID NO: 3 - DNA, 1842 bp, synthetic construct
  source 1..1842: mol_type = other DNA
atgattccgagcaagaagaatgcggttctggtggatggagtcgtgctcaatggaccgactacagatgccaaagcaggcgaaaagttcgtcgaagaggcctgtcgcctgatcatggaggaagtggtactcaaagccaccgatgttaacgaaaaagtttgtgaatggcgtcctccagaacagctgaaacagctgctggatctggaaatgcgcgattcgggtgaaccgccacacaaactgctcgagttgtgtcgtgacgtgatccattattccgtgaaaacgaatcatccgcgcttctttaaccagctctacgccggtttggactattacagcctcgttgctcgctttatgaccgaagccttaaatccgtcggtctacacctatgaggtaagcccggttttcctgctggtagaagaagcggtgttgaaaaagatgattgagttcattgggtggaaagaaggggatggcattttcaacccaggtggtagtgtcagcaacatgtatgcgatgaatttggcgcgctacaagtactgcccggacatcaaagagaaaggccttagtggcagtcctcgtctgatcctctttacttcggcggaatgccactacagcatgaaaaaagccgcgagctttctggggattgggacagaaaacgtgtgttttgtggaaactgacggtcgtggcaaaatgattcctgaagaactggaaaaacaggtgtggcaagcgcgcaaagaaggagcagctccctttctggtgtgtgcaacctccggcacgaccgttttgggcgcatttgatccccttgatgaaattgccgatatctgcgaacgccattccctgtggctgcatgttgacgcgtcttggggtggttcagctctgatgtctcgtaaacaccgcaaacttctgcatggcattcatcgggcagattccgttgcttggaatccgcacaaaatgctgatggctggtattcagtgctgtgcactgctggtgaaagacaaatctgacttactgaagaaatgctatagtgcaaaagcgtcttacttatttcagcaggataagttctacgatgtctcctatgataccggcgacaaatcgatccagtgctcacgtcgtccagatgccttcaaattctggatgacctggaaggcgttgggcacgttaggcctggaggaacgtgtgaatcgcgcgttagcgctgagtcgctatctggtagatgagatcaaaaaacgtgaaggctttaagttgctgatggaacctgagtatgcgaacatctgcttttggtatattccgccctcacttcgtgaaatggaggaaggtccggaattttgggccaaacttaaccttgtagccccggctattaaagagcggatgatgaaaaaaggctcattaatgctggggtatcaaccgcatcgcggtaaagtcaacttctttcgccaagtggtcattagcccgcaagtttcgcgcgaggatatggactttctgctggatgaaatcgatttactgggtaaggacatgtaatctagagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaac

SEQ ID NO: 4 - protein, 502 aa, Homo sapiens
MIPSKKNAVLVDGVVLNGPTTDAKAGEKFVEEACRLIMEEVVLKATDVNEKVCEWRPPEQLKQLLDLEMRDSGEPPHKLLELCRDVIHYSVKTNHPRFFNQLYAGLDYYSLVARFMTEALNPSVYTYEVSPVFLLVEEAVLKKMIEFIGWKEGDGIFNPGGSVSNMYAMNLARYKYCPDIKEKGLSGSPRLILFTSAECHYSMKKAASFLGIGTENVCFVETDGRGKMIPEELEKQVWQARKEGAAPFLVCATSGTTVLGAFDPLDEIADICERHSLWLHVDASWGGSALMSRKHRKLLHGIHRADSVAWNPHKMLMAGIQCCALLVKDKSDLLKKCYSAKASYLFQQDKFYDVSYDTGDKSIQCSRRPDAFKFWMTWKALGTLGLEERVNRALALSRYLVDEIKKREGFKLLMEPEYANICFWYIPPSLREMEEGPEFWAKLNLVAPAIKERMMKKGSLMLGYQPHRGKVNFFRQVVISPQVSREDMDFLLDEIDLLGKDM

SEQ ID NO: 5 - DNA, 1834 bp, synthetic construct
  source 1..1834: mol_type = other DNA
atgagttcgtccaaggaatatatgaatggccacgtgcatctggaggagagtgatatgtatgaatccgatggcaaactgtttctgacggaagcctttaacgttatcatggaagaaattttgcacaaaggtaccgattcggaagagaaagtgtgtgagtggaaagatcccgatcagctgcgtgccctcttggatctggatctgcgtgaacatggggaatcacataaacagctgcttcaacgcgtgcgtgacgttgcacgctattctgtcaaaacctgtcatccgcgcttcttcaatcagctgtttgccggcgtagactatcacgctcttaccggacgcttgctgaccgaaaccctgaatacgagtcagtacacttacgaagtcgctccggtgtttgttcttatggaagaggaagtgctttgcaaattacgcagcctcgtcggatggtctgaaggcgacggtattttctgtcctggcggcagcatgtccaacatgtatgcgatgaatgtggctcgttattgggctttccctcaggttaaaacgcagggtttatgggcaacgccacgtatggcgattttcacctctcaacagggccattacagcatgaagaaagcggcagcctttctgggtattggcacagacaatgtgttcatcgtacaggtcgatgaaagcgggagtatgcgtcctgaggatctcgaagcgaaaatcgtacaagcgaaaagccaggatgccgttcccttctttgtgaacgcaacagccggtacaaccgttcaaggtgcctttgatccgctgaatcgtattgcggacatctcagaacgcaacggcatgtggatgcacgtcgatgcggcttggggtggttcagtgctgtttagcaagaagcacaaacatctggttgcgggcattgaacgtgcaaacagcgtgacttggaatccgcacaaaatgctgctggccgggttacagtgttctgtcgtactgtttcgcgataccacgaatttactgatgcactgccattcggccaaagcgacgtacctcttccaacaagacaagttttacgacacttcgcttgatactggggacaaaagcattcagtgcggtcgcaaggtggattgcttgaaactgtggctgatgtggaaagcgattggcacccatgggttatccgagcgtgtagagaaagcgtttgctttagcacgctatctggtcgaagaaatggaaaagcgcgacaacttcaagctggtgtgcaaaggtccgtttgtaaacgtgtgcttttggtttatcccgccgtcactccgccgcaaagaaaactccgcggattaccaagaacggttgtcgaaagtggcaccggttatcaaagagcggatgatgaaacgtggaacgatgatggtgggttatcagccaatggatggccatgtcaacttctttcgcatggttgttgtcagtccacagttgaccaccaaagacatggacttcttcctggatgagattgaagatctgggcaaagacctgtaagcttgcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaac

SEQ ID NO: 6 - protein, 500 aa, Cyprinus carpio
MSSSKEYMNGHVHLEESDMYESDGKLFLTEAFNVIMEEILHKGTDSEEKVCEWKDPDQLRALLDLDLREHGESHKQLLQRVRDVARYSVKTCHPRFFNQLFAGVDYHALTGRLLTETLNTSQYTYEVAPVFVLMEEEVLCKLRSLVGWSEGDGIFCPGGSMSNMYAMNVARYWAFPQVKTQGLWATPRMAIFTSQQGHYSMKKAAAFLGIGTDNVFIVQVDESGSMRPEDLEAKIVQAKSQDAVPFFVNATAGTTVQGAFDPLNRIADISERNGMWMHVDAAWGGSVLFSKKHKHLVAGIERANSVTWNPHKMLLAGLQCSVVLFRDTTNLLMHCHSAKATYLFQQDKFYDTSLDTGDKSIQCGRKVDCLKLWLMWKAIGTHGLSERVEKAFALARYLVEEMEKRDNFKLVCKGPFVNVCFWFIPPSLRRKENSADYQERLSKVAPVIKERMMKRGTMMVGYQPMDGHVNFFRMVVVSPQLTTKDMDFFLDEIEDLGKDL

SEQ ID NO: 7 - DNA, 457 bp, Escherichia coli
  source 1..457: mol_type = genomic DNA
tccggaataccgtcagggcgtggtgaccgataatggcaacgtgatcctcgacgtccacggcatggaaatccttgacccgatagcgatggaaaacgccataaatgcgattcctggcgtggtgactgttggcttgtttgctaaccgtggcgcggacgttgcgctgattggcacacctgacggtgtcaaaaccattgtgaaatgatctgacgggggaacctcccccgttaaaaaaattctcttcattaaatttggtgacatgtgtcacgtttttacctggctattgtcgattgctctaaataaatcttctaaaccagcatattcatccaagaattacctttgcgtgatatttcctcaacatcgcgacgcaaacgttcatattgccgcaatattattttttgatatgttgaaaggcggacgcaaatccgcacacaacatttcaaaagacaggattgggtaa

SEQ ID NO: 8 - DNA, 65 bp, synthetic construct
  source 1..65: mol_type = other DNA
gtaaattgatcaagtattctgactaagttaaggaggaaattatatggaacgcaccgaactgctga

SEQ ID NO: 9 - DNA, 65 bp, synthetic construct; identical to SEQ ID NO: 8
  source 1..65: mol_type = other DNA

SEQ ID NO: 10 - DNA, 57 bp, synthetic construct
  source 1..57: mol_type = other DNA
gctcgttagagaacaactaataaggaggaaattatatgattccgagcaagaagaatg

SEQ ID NO: 11 - DNA, 55 bp, synthetic construct
  source 1..55: mol_type = other DNA
acgcggcgcatctcgggcagcgttgggtcactagttgcttcgcaacgttcaaatc

SEQ ID NO: 12 - DNA, 35 bp, synthetic construct
  source 1..35: mol_type = other DNA
ataatttcctccttattagttgttctctaacgagc

SEQ ID NO: 13 - DNA, 57 bp, synthetic construct
  source 1..57: mol_type = other DNA
gctcgttagagaacaactaataaggaggaaattatatgagttcgtccaaggaatata